(19) United States
(12) Patent Application Publication
(10) Pub. No.: US 2012/0093882 A1
(43) Pub. Date: Apr. 19, 2012

(54) STABLE PHARMACEUTICAL COMPOSITIONS OF DICLOFENAC

(76) Inventors: Sunilendu Bhushan Roy, Ahmedabad (IN); Shafiq Sheikh, Ahmedabad (IN); Jay Kothari, Ahmedabad (IN); Jitendra Patel, Ahmedabad (IN)

(21) Appl. No.: 13/262,987

(22) PCT Filed: Mar. 22, 2010
(86) PCT No.: PCT/IN2010/000167

§ 371 Date: Dec. 28, 2011

(30) Foreign Application Priority Data
Apr. 8, 2009 (IN) 937/MUM/2009

(51) Int. Cl. A61K 9/107; A61K 31/196; A61P 29/00; C07C 229/42; A61P 25/00; B82Y 40/00; B82Y 5/00

(52) U.S. Cl. 424/400; 562/456; 514/567; 977/788; 977/915; 977/840

(57) ABSTRACT

The present invention discloses a stable pharmaceutical composition of diclofenac or salts thereof comprising nano size droplets of diclofenac or salts thereof, wherein the composition exhibits a significant difference in one or both of the rate and extent of absorption of diclofenac or salts thereof as compared to formulation of diclofenac marketed under the trade name Voveran®

### FIELD OF THE INVENTION

The present invention relates to stable pharmaceutical compositions of diclofenac or salts thereof comprising nano size droplets of diclofenac or salts thereof along with other pharmaceutically acceptable excipients. These compositions exhibit excellent photostability, greater permeability, and improved bioavailability leading to enhanced therpapeutic pharmacodynamic activity as compared to existing formulation of diclofenac marketed under the trade name Voveran®. The invention also relates to process for the preparation of such compositions.

### BACKGROUND OF THE INVENTION

Diclofenac is one of the routinely prescribed anti-inflammatory agents available for the management of pain and inflammation. It is marketed as injection, oral immediate release tablets, sustained release tablets and conventional topical formulations. The drug is almost completely absorbed after oral administration but is subjected to 50% hepatic first-pass metabolism.

Although a major portion of commercial diclofenac is available in the form of oral medications, the drug causes serious adverse effects in the gastrointestinal tract. Gastrointestinal bleeding and ulcerations are quite common due to oral diclofenac. Therefore, topical preparations like creams; ointments for external application are being widely used. However, since diclofenac and its salts are scarcely absorbed percutaneously and thereby require higher quantity to be applied topically thus leading to increased frequency of application also. This leads to patient incompliance.

U.S. Pat. No. 5,629,021 relates to micellar nanoparticles and methods of their production.

U.S. Pat. No. 5,894,019 discloses topical compositions comprising lipid and essentially free of emulsifiers and surfactants.

EP Patent No. 1536836B1 discloses conventional topical emulsion gel of diclofenac sodium.

EP Patent No. 506197B1 discloses an aqueous suspension of solid lipid nanoparticles for topical use.

EP Patent No. 671903B1 discloses topical compositions in the form of submicron oil spheres.

International (PCT) Publication No. 2008/051186 describes nanoemulsion compositions having anti-inflammatory activity.

Clinical evidence suggests that topically applied non-steroidal anti-inflammatory drugs (NSAIDs) are safer than and at least as efficacious as oral NSAIDs in the treatment of rheumatic diseases. Adverse drug reactions after topical administration of NSAID use are rare when compared to the incidence of serious GI events associated with oral NSAIDs. However, formulation may have a dramatic impact on depth of penetration at the site of application, retention of drug molecules within the layers of skin, concentrations achieved in the muscle tissue, synovial fluid and in systemic circulation.

Most of the topical preparations contain vehicles comprising permeation enhancers, solvents, and high amount of surfactants to achieve these goals. But use of these agents is harmful, especially in chronic application, as many of them are irritants. Therefore, there exists a need to develop such topical preparations which does not involve use of such agents as described above to facilitate drug permeation through the skin, and still leads to excellent photostability, greater permeability, and improved bioavailability resulting in enhanced therpapeutic pharmacodynamic activity.

The compositions of the invention overcome all the commonly encountered problems as exemplified above.

### SUMMARY OF THE INVENTION

In one general aspect there is provided a stable pharmaceutical composition of diclofenac or salts thereof comprising nano size droplets of diclofenac or salts thereof.

In another general aspect there is provided a stable pharmaceutical composition of diclofenac or salts thereof comprising nano size droplets of diclofenac or salts thereof, wherein the composition exhibits a significant difference in one or both of the rate and extent of absorption of diclofenac or salts thereof as compared to formulation of diclofenac marketed under the trade name Voveran®.

In another general aspect there is provided a stable pharmaceutical composition of diclofenac or salts thereof comprising nano size droplets of diclofenac or salts thereof, wherein the composition retains at least 80% potency of diclofenac or salts thereof after exposure in a photo stability chamber at light intensity of 1.4 million per lux hour at 250 watt per square meter for 46 hours.

The composition of the invention exhibits significantly enhanced photo stability as compared to Voveran®.

In another general aspect there is provided a stable pharmaceutical composition of diclofenac or salts thereof comprising nano size droplets of diclofenac or salts thereof, wherein the composition retains at least 80% potency of diclofenac or salts thereof after 3 months at 40° C./75% RH.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

In another general aspect there is provided a stable pharmaceutical composition of diclofenac or salts thereof comprising nano size droplets of diclofenac or salts thereof, wherein the composition exhibits significantly enhanced flux of diclofenac or salts thereof as compared to formulation of diclofenac marketed under the trade name Voveran®.

In another general aspect there is provided a stable pharmaceutical composition of diclofenac or salts thereof comprising nano size droplets of diclofenac or salts thereof, wherein the composition exhibits significantly greater percent inhibition in paw edema as compared to formulation of diclofenac marketed under the trade name Voveran®.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

D₉₀ particle size of droplets of diclofenac or salts thereof in the compositions of the invention is about 500 nm.

In another general aspect there is provided a method of improving patient compliance by administering a stable pharmaceutical composition of diclofenac or salts thereof comprising nano size droplets of diclofenac or salts thereof, wherein the composition exhibits a decrease in frequency of application as compared to formulation of diclofenac marketed under the trade name Voveran®.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

In another general aspect there is provided a stable pharmaceutical composition of diclofenac or salts thereof comprising nano size droplets of diclofenac or salts thereof, wherein the composition exhibits a flux of at least 40 mcg/sq.cm/hr in 30 minutes.

In another general aspect there is provided a stable pharmaceutical composition of diclofenac or salts thereof comprising nano size droplets of diclofenac or salts thereof, wherein the composition exhibits a percent inhibition in paw edema of at least 40% in 1 hour.

In another general aspect there is provided a stable pharmaceutical composition of diclofenac or salts thereof comprising nano size droplets of diclofenac or salts thereof, wherein the composition exhibits a percent inhibition in paw edema of at least 15% after 5 hour.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

In another general aspect there is provided a stable pharmaceutical composition of diclofenac or salts thereof comprising nano size droplets of diclofenac or salts thereof, wherein the composition exhibits a maximum plasma concentration (Cₘₐₓ) from about 100 ng/ml to about 300 ng/ml.

In another general aspect there is provided a stable pharmaceutical composition of diclofenac or salts thereof comprising nano size droplets of diclofenac or salts thereof, wherein the composition exhibits a time to reach maximum plasma concentration (Tₘₐₓ) from about 5.0 h to about 8.0 h.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

In another general aspect there is provided a stable pharmaceutical-composition of diclofenac or salts thereof comprising nano size droplets of diclofenac or salts thereof, wherein the composition exhibits an area under the plasma concentration time curve (AUC_{0-inf}) from about 1600 h.ng/ml to about 6900 h.ng/ml.

In another general aspect there is provided a stable pharmaceutical composition of diclofenac or salts thereof comprising nano size droplets of diclofenac or salts thereof, wherein the composition is prepared by a process comprising:
a) combining an oily phase comprising diclofenac or salts thereof along with other pharmaceutically acceptable excipients with an aqueous phase to form an emulsion;
b) reducing the particle size of emulsion of step a) to a droplet size having D₉₀ particle size of about 500 nm; and
c) mixing other pharmaceutically acceptable excipients to the emulsion obtained in step b) and converting it into a suitable finished dosage form.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

In another general aspect there is provided a process of preparing stable pharmaceutical composition of diclofenac or salts thereof comprising nano size droplets of diclofenac or salts thereof, the process comprising:
a) combining an oily phase comprising diclofenac or salts thereof along with other pharmaceutically acceptable excipients with an aqueous phase to form an emulsion;
b) reducing the particle size of emulsion of step a) to a droplet size having D₉₀ particle size of about 500 nm; and
c) mixing other pharmaceutically acceptable excipients to the emulsion obtained in step b) and converting it into a suitable finished dosage form.

Embodiments of the pharmaceutical composition may include one or more of the following features. The pharmaceutical composition may further include one or more pharmaceutically acceptable excipients. For example, the pharmaceutically acceptable excipients may include one or more of oils, lipids, stabilizers, surfactants, initiators, thickening agents, and the like.

The details of one or more embodiments of the invention are set forth in the description below. Other features, objects and advantages of the invention will be apparent from the description and claims.

### BRIEF DESCRIPTION OF THE DRAWINGS

FIG. 1: Graphical illustration of the comparative flux achieved with composition of the invention and Voveran® gel.

FIG. 2: Graphical illustration of the comparative change in paw volume achieved with composition of the invention and Voveran® gel.

FIG. 3: Graphical illustration of the comparative percentage inhibition in paw edema versus time achieved with composition of the invention and Voveran® gel.

FIG. 4: Graphical illustration of the comparative plasma levels achieved with composition of the invention and Voveran® gel.

### DETAILED DESCRIPTION OF THE INVENTION

Various studies in the past have shown that diclofenac is a photosensitizing non-steroidal anti-inflammatory drug. Diclofenac when exposed to sunlight degrades. Thus, labels of the marketed products viz. Voveran®, Voltaren® and Solaraze® also advise patients to avoid exposure to sunlight and the use of sunlamps. During one study, diclofenac dissolved in ultra-pure water was exposed to natural midsummer sunlight for a maximum of 145 h. Fast degradation of diclofenac was observed. Phytotoxicity increased after 3.5 h of exposure of diclofenac to sunlight and showed a maximum of six fold enhanced toxicity after 53 h of exposure to sunlight. Several photo transformation products were found during the experiment. The time courses of the relative concentration of transformation products significantly correlated with enhanced phytotoxicity during the experiment, which indicated a high toxicity potential of photo transformation products of diclofenac.

The inventors of the invention have discovered that when diclofenac or salts thereof is formulated into nano size droplets in pharmaceutically acceptable emulgel system which includes optimized ratios of oils and/or emulsifiers, it leads to highly photo stable compositions of diclofenac or salts thereof. Further, such compositions have enhanced permeability characteristics, improved biovailability and greater therapeutic pharmacodynamic effect as compared to conventional formulation of diclofenac marketed under the trade name Voveran®.

The composition of the invention results in immediate and sustained action and covers large surface area with less quantity and good spreadability, non-irritant to skin and mucous membranes, reduced frequency of application leading to improved patient compliance and offers cosmetic benefits like non-stickiness, and non-greasy feel. The pH of the composition of invention is from about 5.0 to 5.5.

Suitable lipids which can be used include one or more of hydrocarbons, fatty alcohols, fatty acids, glycerides or esters of fatty acids with C₁-C₃₆ alkanols. Hydrocarbons may include paraffin or petroleum jelly. Fatty alcohols may include decanol, dodecanol, tetradecanol, hexadecanol or octadecanol. Fatty acids may include C₆-C₂₄ alkanoic acids such as hexanoic acid, octanoic acid, decanoic acid, dodecanoic acid, tetradecanoic acid, hexadecanoic acid, octadecanoic acid, unsaturated fatty acids such as oleic acid and linoleic acid. Glycerides may include olive oil, castor oil, sesame oil, caprylic/capric acid triglyceride or glycerol mono-, di- and tri-esters with palmitic and/or stearic acid. Esters of fatty acids may include C₁-C₃₆ alkanols such as beeswax, carnauba wax, cetyl palmitate, lanolin, isopropyl myristate, isopropyl stearate, oleic acid decyl ester, ethyl oleate and C₆-C₁₂ alkanoic acid esters.

Suitable oils may include one or more of almond oil, apricot seed oil, borage oil, canola oil, coconut oil, corn oil, cotton seed oil, fish oil, jojoba bean oil, lard oil, linseed oil, boiled macadamia nut oil, mineral oil, olive oil, peanut oil, safflower oil, sesame oil, soybean oil, squalane, sunflower seed oil, tricaprylin (1,2,3 trioctanoyl glycerol) and wheat germ oil.

Suitable stabilizers may include one or more of ionic polysorbate surfactant, Tween® 20, Tween® 40, Tween® 60, Tween® 80, Nonylphenol Polyethylene Glycol Ethers, (alkylphenol-hydroxypolyoxyethylene), Poly(oxy-1,2-ethanediyl), alpha-(4-nonylphenol)-omega-hydroxy-, branched (i.e. Tergitol® NP-40 Surfactant), Nonylphenol Polyethylene Glycol Ether mixtures (i.e. Tergitol® NP-70 (70% AQ) Surfactant), phenoxypolyethoxyethanols and polymers thereof such as Triton®, Poloxamer®, Spans®, Tyloxapol®, different grades of Brij, sodium dodecyl sulfate and the like.

Suitable initiators may include one or more of alcohols like C₁-C₁₂ alcohols, diols and triols, glycerol, methanol, ethanol, propanol, octanol, and the like.

In one embodiment, composition of the invention may be prepared by a) combining an oily phase comprising diclofenac or salts thereof along with other pharmaceutically acceptable excipients with an aqueous phase to form an emulsion; b) reducing the particle size of emulsion of step a) to a droplet size having D % particle size of 500 nm; and c) mixing other pharmaceutically acceptable excipients to emulsion obtained in step b) and converting it into a suitable finished dosage form.

The nano size droplets may be produced with reciprocating syringe instrumentation, continuous flow instrumentation, high speed mixing or high pressure homogenization. Small droplets of the nano emulsion may be formed by passing the emulsion through a homogeniser under different pressures ranging from 3,500-21,500 psi. The emulsion may be passed between 4-5 times under the same conditions to get a final D₉₀ droplet size of about 500 nm. The nano droplets formed may be filtered through 0.2 to 0.4 micron filter.

The gel base may be used in the present invention to form a gel matrix for the preparation of nanogel from nanoemulsion. The gel base comprises of one or more of thickening agents.

Suitable thickening agents may include one or more of cellulose polymer, a carbomer polymer, a carbomer derivative, a cellulose derivative, polyvinyl alcohol, poloxamers, polysaccharides and the like.

Suitable dosage form of the invention may include cream, gel, ointment, lotion, and emulsion.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | | |
|---|---|---|
| Sr. No. | Ingredients | % w/w |

Nano emulsion

| | | |
|---|---|---|
| 1 | Diclofenac sodium | 0.5-3 |
| 2 | Ethanol | 5-20 |
| 3 | Menthol | 0.01-1 |
| 4 | Polysorbate 80 (Tween 80) | 2-10 |
| 5 | Glycerol | 5-20 |
| 6 | Soyabean oil | 10-30 |
| 7 | Methyl salicylate | 0.5-5 |
| 8 | Water | 25-75 |

Nano gel

| | | |
|---|---|---|
| 1 | Diclofenac sodium nanoemulsion | 25-50 |
| 2 | Carbopol gel (2% w/w) | 50-100 |

Procedure: Diclofenac sodium and menthol were dissolved in ethanol and tween 80 mixture along with glycerol. This hydroalcoholic phase was mixed with soyabean oil and methyl salicylate. Water was added with stirring to the resulting mixture. The resulting blend was homogenized to reduce the droplet size to D₉₀ particle size of about 250 nm with the help of high pressure homogenization to get the nano emulsion. Carbomer was added to water for hydration and kept overnight to ensure complete hydration. The aqueous dispersion of carbomer was mixed with nano emulsion to get nanogel.

### Example 2

### Flux Experiment

Instrument used: Hansen diffusion cell

Human cadaver skin was mounted on each of the six diffusion cells with help of clamp.

Medium: pH 7.4 Phosphate buffer

Temperature: 32+2 degree C.

Specimen Preparation In three different cells, composition of the invention was placed and in other three cells Voveran® gel was placed.

The quantity was used around 120 mg to cover exposed skin on each diffusion cell.

Automatic Sampling System: It withdraws the samples at predefined time interval.

Time Interval: 15 min, 30 min, 45 min, 1 hr, 2 hr, 4 hr, 8 hr, 12 hr and 24 hr.

The comparative flux achieved by composition of the invention and the marketed formulation (Voveran®) is demonstrated in Table 2 and FIG. 1, which showed significantly higher flux for the composition of the invention.

TABLE 2

| | Flux (micrograms/sq · cm/hour) | |
|---|---|---|
| No. of | | Composition of |
| hours | Voveran ® | the invention |
| 0 | 0.00 | 0.00 |
| 0.3 | 0.78 | 10.71 |
| 0.5 | 0.86 | 46.24 |
| 0.8 | 1.54 | 32.90 |
| 1.0 | 3.22 | 28.08 |
| 2.0 | 2.16 | 11.09 |
| 4.0 | 4.49 | 7.40 |
| 8.0 | 2.61 | 2.99 |
| 12.0 | 3.25 | 2.98 |
| 24.0 | 0.84 | 0.94 |

### Example 3

### In Vivo Efficacy Study of Composition of the Invention

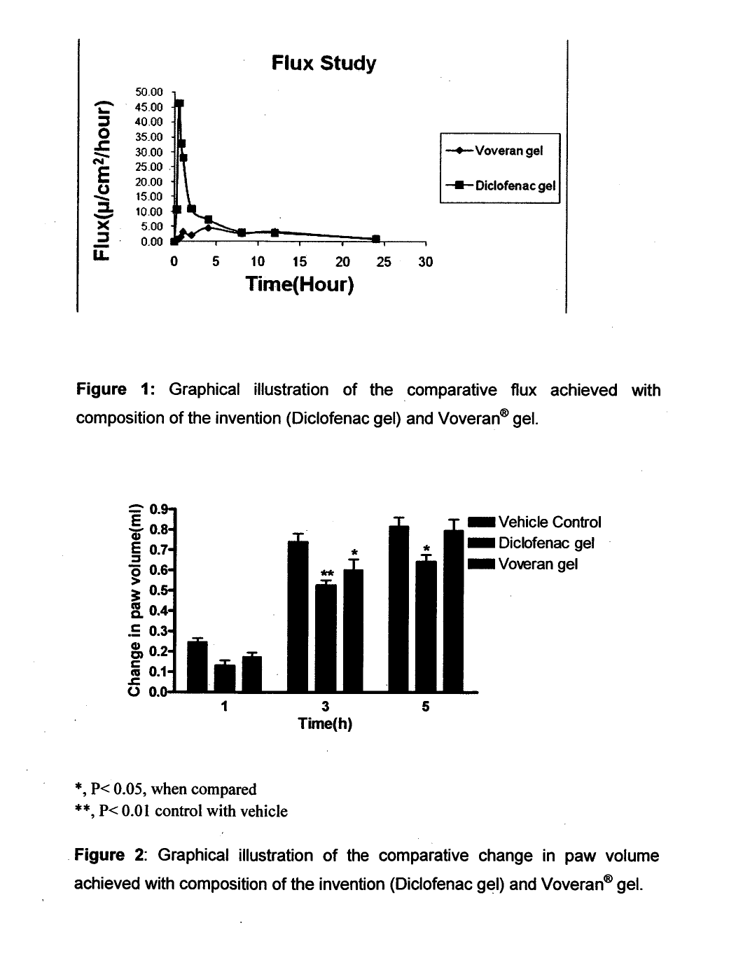

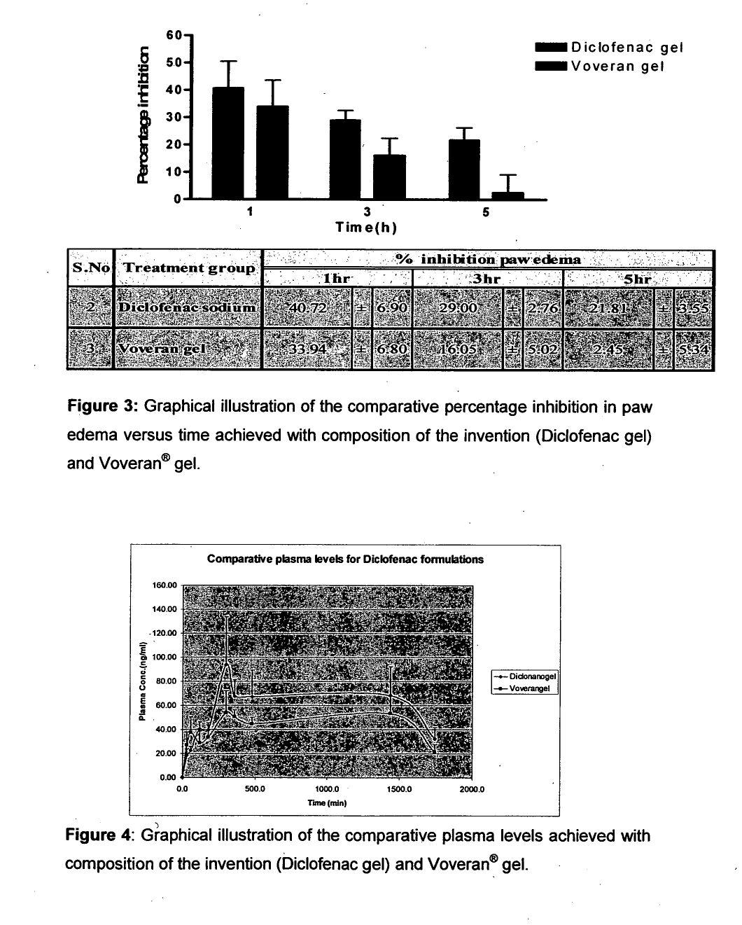

The anti-inflammatory and sustaining action of the optimized formulation was evaluated by the carrageenan-induced paw edema method developed by Winter et al in Wistar rats. Young Wistar rats weighing 120 to 150 g were randomly divided into 3 groups: control, nanogel and Voveran® (Innovator, Novartis), each containing 6 rats. The animals were kept under standard laboratory conditions, with temperature of 25° C.±1° C. and relative humidity of 55%±5%. The animals were housed in polypropylene cages, 6 per cage, with free access to a standard laboratory diet and water ad libitum. The composition of the invention and Voveran® were applied on the paw region of all animals (except in control group) half an hour before subplanter injection of carrageenan in right paws. Paw edema was induced by injecting 0.1 mL of the 1% w/w homogeneous suspension of carrageenan in distilled water. The volume of paw was measured at 1, 3 and 5 hours after injection using a digital plethysmometer. The amount of paw swelling was determined for 5 hours and expressed as percent edema relative to the initial paw volume. The percent inhibition of edema produced by each formulation-treated group was calculated against the respective control group. The results of anti-inflammatory activity were compared using the Dunnett test of 1-way ANOVA. The anti-inflammatory effect of composition of the invention vs Voveran® is shown in FIG. 2. The percentage inhibition as produced is shown in FIG. 3.

The in vivo efficacy study of composition of the invention demonstrated greater percentage inhibition of paw edema compared to the marketed formulation (Voveran®).

### Example 4

The comparative plasma levels achieved by composition of the invention and the marketed formulation (Voveran®) are demonstrated in Tables 3, 4, 5 and FIG. 4.

TABLE 3

| Time point | Composition of | |
|---|---|---|
| (min) | the invention | Voveran ® |
| 0.0 | 0.00 | 0.00 |
| 30.00 | 18.48 | 26.99 |
| 60.00 | 22.45 | 37.60 |
| 120.00 | 40.53 | 28.74 |
| 180.00 | 39.65 | 33.19 |
| 300.00 | 93.62 | 53.88 |
| 360.00 | 67.53 | 50.59 |
| 480.00 | 66.88 | 44.55 |
| 1440.00 | 68.32 | 53.48 |
| 1740.00 | 32.17 | 21.21 |

TABLE 4
Pharmacokinetic Parameters of composition of the invention
in Wistar rats at 100 mg dose (topical application 2 × 3 cm)

| Animal | Tₘₐₓ | Cₘₐₓ | T_{1/2} | Kₑₗ | AUC (0-t) | AUC (0-inf) |
|---|---|---|---|---|---|---|
| No. | (hr) | (ng/ml) | (hr) | (hr−1) | (hr · ng/ml) | (hr · ng/ml) |
| ID-11 | 8.00 | 180.56 | 12.81 | 0.05 | 3894.72 | 4642.92 |
| ID-15 | 5.00 | 114.70 | 9.66 | 0.07 | 1365.62 | 1606.37 |
| ID-16 | 24.00 | 64.20 | — | — | 1478.72 | — |
| ID-17 | 5.00 | 299.34 | 38.86 | 0.02 | 2428.44 | 6855.90 |
| ID-18 | 0.50 | 36.42 | 17.22 | 0.04 | 519.78 | 708.36 |
| ID-19 | 5.00 | 49.43 | 26.55 | 0.03 | 917.25 | 1621.97 |
| Mean | 7.90 | 124.11 | 21.02 | 0.04 | 1767.42 | 3087.10 |
| SD | 9.21 | 100.83 | 11.83 | 0.02 | 1223.64 | 2579.83 |
| S.E.M. | 5.32 | 58.22 | 6.83 | 0.01 | 706.47 | 1489.46 |

TABLE 5
Pharmacokinetic Parameters of Voveran ® gel in wistar
rats at 100 mg dose (topical application 2 × 3 cm)

| Animal | Tₘₐₓ | Cₘₐₓ | T_{1/2} | Kₑₗ | AUC (0-t) | AUC (0-inf) |
|---|---|---|---|---|---|---|
| No. | (hr) | (ng/ml) | (hr) | (hr−1) | (hr · ng/ml) | (hr · ng/ml) |
| ID-14 | 0.00 | 61.08 | 6.32 | 0.11 | 499.16 | 552.14 |
| ID-10 | 0.00 | 140.31 | 8.41 | 0.08 | 1231.28 | 1308.19 |
| ID-7 | 24.00 | 78.39 | — | — | 1471.07 | — |
| ID-2 | 0.00 | 60.10 | 31.78 | 0.02 | 1328.29 | 2744.88 |
| ID-3 | 24.00 | 81.65 | — | — | 1311.31 | — |
| ID-5 | 24.00 | 89.58 | — | — | 1909.35 | — |
| Mean | 12.00 | 85.19 | 15.50 | 0.07 | 1291.74 | 1535.07 |
| SD | 13.15 | 29.42 | 14.13 | 0.04 | 457.64 | 1113.84 |
| S.E.M. | 7.59 | 16.98 | 8.16 | 0.03 | 264.22 | 643.07 |

### Example 5

The photostability studies of the composition of the invention and Voveran® were performed using a specific quantity of the composition of the invention and Voveran® into a glass petriplate and subjecting to a photostability chamber for 46 hours with 1.4 million per lux hour exposure and 250 watt per square meter. The results of single impurity and total impurities are shown in Table 6, which clearly indicates the excellent photostability of the composition of the invention as compared to Voveran®

TABLE 6

| | Single Impurity | | Total Impurity | |
|---|---|---|---|---|
| | | After Exposure | | After Exposure |
| Product | Initial | for 46 hrs | Initial | for 46 hrs |
| Composition of | 0.32% | 1.26% | 0.37% | 1.39% |
| the invention | | | | |
| Voveran ® | 1.02% | 3.06% | 2.55% | 8.57% |

### Example 6

The composition of the invention was also subjected to 3 month and 6 month accelerated stability studies at 40° C./75% RH. The results are shown in Table 7, which clearly indicates excellent stability of the composition of the invention.

TABLE 7

| Composition of | 40° C./75% RH | 40° C./75% RH |
|---|---|---|
| the invention | for 3 months | for 6 months |
| | 98.28% | 97.27% |

We claim:

1. A stable pharmaceutical composition of diclofenac or salts thereof comprising nano size droplets of diclofenac or salts thereof, wherein the composition exhibits a significant difference in one or both of the rate and extent of absorption of diclofenac or salts thereof as compared to formulation of diclofenac marketed under the trade name Voveran®.

2. The composition as claimed in claim 1, wherein the composition exhibits a maximum plasma concentration (Cₘₐₓ) from about 1 ng/ml to about 300 ng/ml.

3. The composition as claimed in claim 1, wherein the composition exhibits a time to reach maximum plasma concentration (Tₘₐₓ) from about 5.0 h to about 8.0 h.

4. The composition as claimed in claim 1, wherein the composition exhibits an area under the plasma concentration time curve (AUC_{0-inf}) from about 1600 h.ng/ml to about 6900 h.ng/ml.

5. The composition as claimed in claim 1, wherein the nano size droplets of diclofenac or salts thereof has a D₉₀ particle size of about 500 nm.

6. The composition as claimed in claim 1, wherein the composition further comprises one or more pharmaceutically acceptable excipients comprising oils, lipids, stabilizers, thickening agents and initiators.

7. A stable pharmaceutical composition of diclofenac or salts thereof comprising nano size droplets of diclofenac or salts thereof, wherein the composition retains at least 80% potency of diclofenac or salts thereof after exposure in a photo stability chamber with light intensity of 1.4 million per lux hour at 250 watt per square meter for 46 hours.

8. The composition as claimed in claim 7, wherein the nano size droplets of diclofenac or salts thereof has a D₉₀ particle size of about 500 nm.

9. The composition as claimed in claim 7, wherein the composition further comprises one or more pharmaceutically acceptable excipients comprising oils, lipids, stabilizers, thickening agents and initiators.

10. A stable pharmaceutical composition of diclofenac or salts thereof comprising nano size droplets of diclofenac or salts thereof, wherein the composition retains at least 80% potency of diclofenac or salts thereof after storage at 3 months at 40° C./75% RH.

11. The composition as claimed in claim 10, wherein the nano size droplets of diclofenac or salts thereof has a D₉₀ particle size of about 500 nm.

12. The composition as claimed in claim 10, wherein the composition further comprises one or more pharmaceutically acceptable excipients comprising oils, lipids, stabilizers, thickening agents and initiators.

13. A stable pharmaceutical composition of diclofenac or salts thereof comprising nano size droplets of diclofenac or salts thereof, wherein the composition exhibits significantly enhanced flux of diclofenac or salts thereof as compared to formulation of diclofenac marketed under the trade name Voveran®.

14. The composition as claimed in claim 13, wherein the nano size droplets of diclofenac or salts thereof has a D₉₀ particle size of about 500 nm.

15. The composition as claimed in claim 13, wherein the composition exhibits a flux of at least 40 mcg/sq.cm/hr in 30 minutes.

16. The composition as claimed in claim 13, wherein the composition further comprises one or more pharmaceutically acceptable excipients comprising oils, lipids, stabilizers, thickening agents and initiators.

17. A stable pharmaceutical composition of diclofenac or salts thereof comprising nano size droplets of diclofenac or salts thereof, wherein the composition exhibits significantly greater percent inhibition of paw edema as compared to formulation of diclofenac marketed under the trade name Voveran®.

18. The composition as claimed in claim 17, wherein the nano size droplets of diclofenac or salts thereof has a D₉₀ particle size of about 500 nm.

19. The composition as claimed in claim 17, wherein the composition exhibits a percent inhibition in paw edema of at least 40% in 1 hour.

20. The composition as claimed in claim 17, wherein the composition exhibits a percent inhibition in paw edema of at least 15% after 5 hours.

21. The composition as claimed in claim 17, wherein the composition further comprises one or more pharmaceutically acceptable excipients comprising oils, lipids, stabilizers, thickening agents and initiators.

22. A method of improving patient compliance by administering a stable pharmaceutical composition of diclofenac or salts thereof comprising nano size droplets of diclofenac or salts thereof, wherein the composition exhibits a decrease in frequency of application as compared to existing formulation of diclofenac marketed under the trade name Voveran®.

23. The method as claimed in claim 22, wherein the nano size droplets of diclofenac or salts thereof has a D₉₀ particle size of about 500 nm.

24. The method as claimed in claim 22, wherein the composition further comprises one or more pharmaceutically acceptable excipients comprising oils, lipids, stabilizers, thickening agents and initiators.

25. A stable pharmaceutical composition of diclofenac or salts thereof comprising nano size droplets of diclofenac or salts thereof, wherein the composition is prepared by a process comprising:
a) combining an oily phase comprising of diclofenac or salts thereof along with other pharmaceutically acceptable excipients with an aqueous phase to form an emulsion;
b) reducing the particle size of emulsion of step a) to a droplet size having D₉₀ particle size of about 500 nm; and
c) mixing other pharmaceutically acceptable excipients to the emulsion obtained in step b) and converting it into a suitable finished dosage form.

26. The composition as claimed in claim 25, wherein the composition further comprises one or more pharmaceutically acceptable excipients comprising oils, lipids, stabilizers, thickening agents and initiators.

27. A process of preparing a stable pharmaceutical composition of diclofenac or salts thereof comprising nano size droplets of diclofenac or salts thereof, the process comprising:
a) combining an oily phase comprising of diclofenac or salts thereof along with other pharmaceutically acceptable excipients with an aqueous phase to form an emulsion;
b) reducing the particle size of emulsion of step a) to a droplet size having D₉₀ particle size of about 500 nm; and
c) mixing other pharmaceutically acceptable excipients to the emulsion obtained in step b) and converting it into a suitable finished dosage form.

28. The process as claimed in claim 27, wherein the composition further comprises one or more pharmaceutically acceptable excipients comprising oils, lipids, stabilizers, thickening agents and initiators.
